(19) United States
(12) Patent Application Publication
(10) Pub. No.: US 2006/0193923 A1
(43) Pub. Date: Aug. 31, 2006

(54) Compositions and methods for increasing muscle mass and strength, improving athletic performance, and/or reducing body fat mass leading to weight loss

(76) Inventors: Paul T. Gardiner, Mississauga (CA); Marvin A. Heuer, Mississauga (CA); Brad H. Pilon, Mississauga (CA)

Correspondence Address: KENYON & KENYON LLP, ONE BROADWAY, NEW YORK, NY, 10004, US

(21) Appl. No.: 11/361,799

(22) Filed: Feb. 24, 2006

Related U.S. Application Data
(60) Provisional application No. 60/657,615, filed on Feb. 28, 2005.

(51) Int. Cl. A61K 36/87; A61K 36/537; A61K 33/32; A61K 31/22; A61K 36/48

(52) U.S. Cl. 424/641; 424/746; 424/766; 424/725; 514/350; 514/546; 424/757

(57) ABSTRACT

Methods and compositions for the inhibition of human aromatase enzyme are presented for the purposes of increasing or activating the total levels of testosterone, free testosterone, and/or luteinizing hormone levels, while decreasing the levels of estrogen and estradiol. The increase in testosterone levels, combined with a proper exercise and diet regime may lead to increased muscle mass and strength, improved athletic performance and recovery and lead to an increase in lean body mass. Red Wine extracts containing Proanthocyanidins are employed to inhibit the action of the human aromatase enzyme.

### RELATED APPLICATIONS

The application is related to and claims benefit of priority to U.S. Provisional Patent Application Ser. No. 60/657,615 entitled “Compositions and Methods for Increasing Muscle Mass and Strength, Improving Athletic Performance, and/or Reducing Body Fat Mass Leading to Weight Loss,” filed Feb. 28, 2005, the disclosure of which is hereby fully incorporated by reference.

### FIELD OF THE INVENTION

The present invention concerns compositions and methods for inhibiting the human aromatase enzyme to increase or activate total testosterone, free testosterone, and/or luteinizing hormone levels, for the purposes of increasing muscle mass and strength, improving athletic performance and recovery therefrom, and/or reducing body fat mass leading to weight loss.

In a preferred embodiment, the present invention concerns the use of red wine and its extracts to inhibit the aromatase enzyme within the human body, resulting in an increase in, or activation of, total testosterone, free testosterone and/or luteinizing hormone levels while simultaneously achieving a reduction in estrogen and/or estradiol levels, for the purposes of increasing muscle mass and strength, improving athletic performance and recovery therefrom, and/or reducing body fat mass leading to weight loss.

### SUMMARY OF THE INVENTION

The present invention, according to one embodiment thereof, concerns compositions and methods for inhibiting the human aromatase enzyme to increase or activate total testosterone, free testosterone, and/or luteinizing hormone levels, for the purposes of increasing muscle mass and strength, improving athletic performance and recovery therefrom, and/or reducing body fat mass leading to weight loss.

In a preferred embodiment, the present invention concerns the use of red wine and its extracts to inhibit the aromatase enzyme within the human body, resulting in an increase in or activation of total testosterone, free testosterone and/or luteinizing hormone levels while simultaneously achieving a reduction in estrogen and/or estradiol levels for the purposes of increasing muscle mass and strength, improving athletic performance and recovery therefrom, and/or reducing body fat mass leading to weight loss.

In addition or alternatively, the present invention provides, according to various embodiments thereof, compositions and methods for increasing muscle mass and strength, improving athletic performance and recovery therefrom, and/or reducing body fat mass leading to weight loss, by increasing or activating total testosterone, free testosterone and/or luteinizing hormone levels while simultaneously reducing estrogen and/or estradiol levels.

In addition or alternatively, the present invention provides, according to various embodiments thereof, compositions and methods for improving memory and lean muscle mass, positively affecting mood, and/or the prevention of sarcopenia and/or other disorders commonly associated with aging.

### DETAILED DESCRIPTION OF THE INVENTION

The present invention, according to various embodiments thereof, provides compositions and methods for increasing muscle mass and strength, improving athletic performance and recovery therefrom, and/or reducing body fat mass leading to weight loss. More specifically, the present invention provides for the use of red wine and its extracts to inhibit the human aromatase enzyme, resulting in an increase in or activation of total testosterone, free testosterone, and/or luteinizing hormone levels and a simultaneous decrease in estrogen and/or estradiol levels for the purposes of increasing muscle mass and strength, improving athletic performance and recovery therefrom, and/or reducing body fat mass leading to weight loss.

As a metabolite of testosterone, within the testes, a percentage of testosterone is converted to estradiol via aromatization (Harper's Biochemistry 25^{th} ed. Murray et al., 2000, Appleton and Lange, Toronto). Aromatase is the enzyme responsible for the conversion of androgen substrates, such as testosterone into estrogens. Proanthocyanidins from red wine extracts been shown to inhibit the action of aromatase activity (Eng E T, et al. Cancer Res. 2003 Dec. 1;63(23):8516-22). Therefore, the actions of proanthocyanidins inhibiting the human aromatase enzyme inhibits the conversion of testosterone to estradiol, thus leading to increased levels of free testosterone within the body. The use of red wine extracts to inhibit the production of estradiol has also been employed in treatment of breast cancers.

The compositions of the present invention comprise red wine and/or its extracts. Certain polyphenols found in red wine, particularly Pinot Noir, Merlot, and Cabernet Sauvignon, may inhibit the human aromatase enzyme in a dose dependent manner. According to one embodiment, the present invention may employ extracts from red grapes and white button mushroom phytochemicals, as well as extracts of Broussonetia Papyrifera. In contrast, white wine and its extracts do not contain all of the same compounds as red wine and do not appear to have the ability to decrease the activity of aromatase, thus the present invention may provide further and significant inhibition of aromatase enzyme. Aromatase enzymes may catalyze estrogen biosynthesis. Inhibition of aromatase with pharmaceutical non-steroidal aromatase inhibitors in normal healthy men may cause an increase in, or activation of, total testosterone, free testosterone and/or luteinizing hormone, with a subsequent decrease in estrogen and/or estradiol for the purposes of increasing muscle mass and strength, improving athletic performance and recovery therefrom, and/or reducing body fat mass leading to weight loss.

Although the present invention is not to be limited by any theoretical explanation, it is believed that inhibition of aromatase is a primary factor that increases or activates testosterone levels while simultaneously decreasing estrogen levels, and that certain polyphenols found in red wine and its extracts may inhibit aromatase activity. It is believed that red wine and/or its extracts have a similar ability to increase or activate total testosterone, free testosterone and luteinizing hormone, with a subsequent decrease in estrogen and estradiol. It is believed that the increased or activated testosterone and/or luteinizing hormone levels lead to improvements in muscle mass and strength, athletic performance and recovery therefrom, and reductions in body fat mass leading to weight loss.

According to the invention there is provided a composition for increasing muscle mass and strength, improving athletic performance and recovery therefrom, and/or reducing body fat mass leading to weight loss, the composition comprising red wine and/or its extract. Suitable red wines and extracts thereof include, but are not limited to, Pinot Noirs, Merlots, and Cabernet Sauvignons. Preferably, the composition is in the nature of a dietary supplement.

The supplement compositions according to the present invention may be employed in methods for increasing muscle mass and strength, improving athletic performance and recovery therefrom, and/or reducing body fat mass leading to weight loss by causing an inhibition of aromatase activity. The compositions of the present invention are particularly advantageous for athletes and bodybuilders to enhance performance. The amount of the composition which is administered to the athlete may vary depending on the desired effect, the body weight and characteristics of the athlete, and the like. For example, in preferred embodiments, the subject compositions comprising red wine and/or its extracts are administered to the diet of the athlete or bodybuilder on a daily basis.

The ability to increase or activate total testosterone, free testosterone, and/or luteinizing hormone levels while simultaneously decreasing estradiol and/or estrogen levels may also be desired in individuals having relatively low testosterone levels, for example hypogonadal older men.

According to a further aspect of the invention there is provided a method for improving memory and lean muscle mass, positively effecting mood, and/or for the prevention of sarcopenia and other disorders commonly associated with aging. This is particularly desired, for example, by the aging male population in the prevention of disorders commonly associated with aging.

Example 1 provides, in accordance with one embodiment of the present invention, a continuous, 24 hour testosterone support formulation. In this embodiment, the formulation provides for two supplemental dietary compositions, e.g., a daytime supplemental dietary composition and a night-time supplemental dietary composition, although any number of formulations may be provided throughout the day. Advantageously, both the day-time and night-time formulations of the supplemental dietary composition may include at least Eurycoma Longifolia, Grape Skin Extract and Red Wine Extract to provide proanthocyanidins. In addition, the night-time formulation may also include a blend of Zinc, Magnesium and Vitamin B6. Also, the day-time formulation may include Tibulus terrestris supplying Saponins, Acetyl-L-Carnitine, Xanthinol Nicotinate and/or Clary Sage Extract, supplying Sclareolides.

This dual-action formulation provides for improved testosterone support throughout the day by maintaining healthy testosterone levels during the a.m. and p.m. hours. It may do this by responding to male hormonal changes that occur naturally throughout the day. For example, in the morning, when testosterone levels are typically higher, the a.m. formulation helps reduce the conversion of testosterone to estrogen. Later in the day, when testosterone levels are typically reduced and are at their lowest levels, the p.m. formula works to increase testosterone levels. In this manner, testosterone levels may be maintained and supported more effectively throughout the day.

Although the following examples illustrate the practice of the present invention in some of its embodiments, the examples should not be construed as limiting the scope of the invention. Other embodiments will be apparent to one skilled in the art from consideration of the specification and examples.

### EXAMPLES

Example 1

| | | Active | |
|---|---|---|---|
| Ingredients (PM | 2.710 | Constituents | |
| Formulation) | g/serving | g/serving | Formula % |
| ZMA Blend (Zinc, Mg, | 2.400 | 0.0324*Zn | 88.5609% |
| Vitamin B6) | | 0.490*Mg | |
| | | 0.01248*Vitamin | |
| | | B6 | |
| Grape Skin Extract | 0.050 | 0.01 | 1.8450% |
| (std. to 20% | | Proanthocyanidins | |
| Proanthocyanidins) | | | |
| Red Wine Extract (std. to | 0.250 | 0.0625 | 9.2251% |
| 25% Proanthocyanidins) | | Proanthocyanidins | |
| Eurycoma Longifolia | 0.010 | | 0.3690% |
| (50:1 Extract from root) | | | |
| Total | 2.710 | | 100% |
| | | Active | |
| Ingredients (AM | 2.235 | Constituents | |
| Formulation | g/serving | g/serving | Formula % |
| Tibulus terrestris from | 0.100 | 0.045 Saponins | 4.4743% |
| whole plant | | | |
| (45% Saponins) | | | |
| Acetyl-L-Carnitine | 0.500 | | 22.3714% |
| Grape Skin Extract | 0.50 | 0.05 | 11.1857% |
| (std. to 20% | | Proanthocyanidins | |
| Proanthocyanidins) | | | |
| Red Wine Extract (std. to | 1.250 | 0.3125 | 55.9284% |
| 25% Proanthocyanidins) | | Proanthocyanidins | |
| Eurycoma Longifolia | 0.010 | | 0.4474% |
| (50:1 Extract from root) | | | |
| Xanthinol Nicotinate | 0.100 | | 4.4743% |
| Clary Sage Extract from | 0.025 | 0.0025 | 1.1186% |
| Whole plant (10% | | Sclareolides | |
| Sclareolides) | | | |
| Total | 2.235 | | 100% |

| | Ingredient | g/serving |
|---|---|---|
| Example 2 | Red Wine Extract | 0.500 |
| Example 3 | Tribulus terrestris | 0.100 |
| | Acetyl-L-Carnitine | 0.500 |
| | Grape Seed Extract | 0.500 |
| | Red Wine Extract | 1.250 |
| | Coleus Forskhoii | 0.025 |
| | Eurycoma Longifolia | 0.020 |
| | Clary Sage Extract 10% | 0.025 |
| Example 4 | ZMA Blend | 2.400 |
| | Grape Seed Extract | 0.500 |
| | Red Wine Extract | 0.250 |
| | Eurycoma Longifolia | 0.020 |
| Example 5 | Creatine Monohydrate | 3.000 |
| | Melatonin | 0.030 |
| | Red Wine Extract | 0.500 |
| Example 6 | Creatine Monohydrate | 5.000 |
| | Alpha Lipoic Acid | 0.250 |
| | Red Wine Extract | 0.250 |
| Example 7 | Red Wine Extract | 0.050 |
| | Grape skin Extract | 0.050 |
| | Coenzyme Q10 | 0.025 |
| | Astaxanthin | 0.025 |
| | Pycnogenol | 0.005 |
| | N-Acetyl-Cysteine | 0.012 |
| | Vitamin C | 0.012 |
| Example 8 | Red Wine Extract | 0.100 |
| | Whey Protein | 20.000 |
| | Creatine Monohydrate | 3.000 |
| Example 9 | Red Wine extract | 0.150 |
| | Green Tea Extract | 0.200 |
| | Caffeine | 0.200 |
| Example 10 | Red wine Extract | 0.400 |
| | Di-indole Methane | 0.170 |
| | Chrysin | 0.100 |
| | Coleus Forskholii | 0.250 |
| Example 11 | Delta-4-1012-dimethyl-cyclopent- | 0.300 |
| | a-phenantrene-3617-trion | |
| | Red Wine Extract | 0.300 |

What is claimed:

1. A supplemental dietary composition for at least one of reducing the conversion of testosterone to estrogen, increasing luteinizing hormone levels, and increasing free testosterone levels, the composition comprising:
Eurycoma Longifolia;
Grape Skin Extract; and
Red Wine Extract to provide proanthocyanidins.

2. The supplemental dietary composition of claim 1, wherein a day-time and a night-time formulation are provided separately.

3. The supplemental composition of claim 2, wherein the night-time formulation further comprises a blend of Zinc, Magnesium and Vitamin B6.

4. The supplemental composition of claim 2, wherein the daytime formulation further comprises Tibulus terrestris supplying Saponins.

5. The supplemental composition of claim 2, wherein the daytime formulation further comprises Acetyl-L-Carnitine.

6. The supplemental composition of claim 2, wherein the daytime formulation further comprises Xanthinol Nicotinate.

7. The supplemental composition of claim 2, wherein the daytime formulation further comprises Clary Sage Extract, supplying Sclareolides.

8. A method for at least one of reducing the conversion of testosterone to estrogen, increasing luteinizing hormone levels, and increasing free testosterone levels, the method comprising the steps of:
ingesting a night-time supplemental dietary composition that includes at least Eurycoma Longifolia, Grape Skin Extract and Red Wine Extract to provide proanthocyanidins.

9. A method for at least one of reducing the conversion of testosterone to estrogen, increasing luteinizing hormone levels, and increasing free testosterone levels, the method comprising the steps of:
ingesting a daytime supplemental dietary composition that includes at least Eurycoma Longifolia, Grape Skin Extract and Red Wine Extract to provide proanthocyanidins.

10. The method of claim 8, wherein the night-time formulation further comprises a blend of Zinc, Magnesium and Vitamin B6.

11. The method of claim 9, wherein the daytime formulation further comprises Tibulus terrestris supplying Saponins.

12. The method of claim 9, wherein the daytime formulation further comprises Acetyl-L-Carnitine.

13. The method of claim 9, wherein the daytime formulation further comprises Xanthinol Nicotinate.

14. The method of claim 9, wherein the daytime formulation further comprises Clary Sage Extract, supplying Sclareolides.

15. The method of claim 8, wherein the Eurycoma Longifolia, Grape Skin Extract and Red Wine Extract providing proanthocyanidins are present in an amount effective to at least one of reduce the conversion of testosterone to estrogen, increase luteinizing hormone levels and increase free testosterone levels.

16. The method of claim 9, wherein the Eurycoma Longifolia, Grape Skin Extract and Red Wine Extract providing proanthocyanidins are present in an amount effective to at least one of reduce the conversion of testosterone to estrogen, increase luteinizing hormone levels and increase free testosterone levels.

17. A method for providing a continuous, 24 hour testosterone support formulation by at least one of reducing the conversion of testosterone to estrogen, increasing luteinizing hormone levels, and increasing free testosterone levels, the method comprising the steps of:
ingesting a daytime supplemental dietary composition that includes at least Eurycoma Longifolia, Grape Skin Extract and Red Wine Extract to provide proanthocyanidins,
ingesting a night-time supplemental dietary composition that includes at least Eurycoma Longifolia, Grape Skin Extract and Red Wine Extract to provide proanthocyanidins.

18. A continuous, 24 hour testosterone support formulation comprising:
a daytime supplemental dietary composition that includes at least Eurycoma Longifolia, Grape Skin Extract and Red Wine Extract to provide proanthocyanidins,
a night-time supplemental dietary composition that includes at least Eurycoma Longifolia, Grape Skin Extract and Red Wine Extract to provide proanthocyanidins.
